(19) United States
(12) Patent Application Publication
(10) Pub. No.: US 2025/0171384 A1
(43) Pub. Date: May 29, 2025

(54) ENERGY-EFFICIENT PROCESS FOR SEPARATION OF 1-BUTENE FROM A HYDROCARBON STREAM

(71) Applicant: Evonik Oxeno GmbH & Co. KG, Marl (DE)

(72) Inventors: Niklas Paul, Marl (DE); Armin Matthias Rix, Marl (DE); Moritz Schröder, Muenster (DE); Tanita Valèrie Six, Dortmund (DE); Tanja Steenweg, Dortmund (DE); Wim Mellaerts, Hoevenen (BE); Johannes Knossalla, Everswinkel (DE); Stephan Peitz, Oer-Erkenschwick (DE); Guido Stochniol, Haltern am See (DE); Tobias Laiblin, Brasschaat (BE)

(73) Assignee: Evonik Oxeno GmbH & Co. KG, Marl (DE)

(21) Appl. No.: 18/958,063

(22) Filed: Nov. 25, 2024

(30) Foreign Application Priority Data
Nov. 29, 2023 (EP) 23213045.0

(51) Int. Cl. C07C 7/04

(52) U.S. Cl. CPC C07C 7/04

(57) ABSTRACT

A process separates 1-butene from a hydrocarbon stream containing at least 1-butene, 2-butene, n-butane and isobutane in a separation unit having at least two distillation columns DK1 and DK2, wherein the heat of condensation is utilized in order to save energy costs and reduce CO₂ emission.

### CROSS-REFERENCE TO RELATED APPLICATIONS

This patent application claims priority to European Patent Application No. 23213045.0, filed on Nov. 29, 2023, in the European Patent Office, the entire disclosure of which is hereby incorporated by reference herein.

### BACKGROUND OF THE INVENTION

### Field of the Invention

The present invention relates to a process for separating 1-butene from a C4 hydrocarbon stream containing at least 1-butene, 2-butene, n-butane and isobutane in a separation unit comprising at least two distillation columns DK1 and DK2, wherein the heat of condensation is rendered utilizable in order to save energy costs and reduce CO₂ emission.

1-Butene can be obtained in large amounts from technical C4 hydrocarbon streams, for example the C4 cut from steamcrackers or FCC units. These C4 hydrocarbon streams consist essentially of butadiene, the monoolefins isobutene, 1-butene and the two 2-butenes (cis-and trans-2-butene), and the saturated hydrocarbons isobutane and n-butane. Because of the small differences in the boiling points of the constituents, the low separation factors thereof and the formation of azeotropes, exclusively distillative workup of C4 hydrocarbon streams is difficult and uneconomic.

Typically, therefore, the butadiene is first separated off by extractive distillation or hydrogenated selectively to butenes. What remains in each case is a C4 hydrocarbon stream (called raffinate 1) containing not only the saturated hydrocarbons n-butane and isobutane but also the olefins isobutene, 1-butene and 2-butenes, while the butadiene is present in small amounts at most.

Since the boiling points of 1-butene and isobutene are close to one another, it is generally not possible to economically separate 1-butene from corresponding C4 hydrocarbon streams via simple distillation. The isobutene is therefore removed as far as possible, for example via an MTBE or ETBE synthesis. The removal of the isobutene gives rise to a C4 hydrocarbon stream (called raffinate 2) containing the linear butenes (1-and 2-butenes) and the saturated hydrocarbons isobutane and n-butane.

### Description of Related Art

The separation of 1-butene from such C4 hydrocarbon streams is possible and is used in the chemical industry. This separation is effected in a distillation unit comprising at least two distillation columns. Isobutane and 1-butene are obtained at the top of the first distillation column and directed to the second distillation column. Then, isobutane and 1-butene are separated from one another in the second distillation column. Such a process is disclosed, for example, in DE 10 2005 062 700 A1.

In the known processes, the energy needed for the separation of the C4 hydrocarbon stream is typically introduced into the bottom of the two distillation columns via heating steam. Heating steam is generally available at chemical production sites. In the volumes needed for the separation tasks in question, the use of heating steam means a cost factor that cannot be underestimated. Moreover, recycling of the spent heating steam is not always simple in logistic terms since the steam can only be returned within certain specifications (pressure, temperature, or the like). Furthermore, the generation of heating steam generates a large volume of CO₂.

### SUMMARY OF THE INVENTION

It was therefore an object of the present invention to provide a process in which energy and CO₂ emission can be saved by comparison with the known processes and which can be integrated into existing plants.

### BRIEF DESCRIPTION OF THE DRAWINGS

FIG. 1 shows a configuration according to a conventional dual pressure circuit.

FIG. 2 shows a configuration in which stream BS1a is brought to a higher pressure with compressor V1, then directed to reboiler SV1a for energy transfer, and the two streams BS1a and BS1b are combined after the energy transfer and go partly to DK1 as reflux and partly to DK2 as feed.

FIG. 3 shows a configuration in which the stream compressed with compressor V1 is called VB1. In addition, vapour stream BS2 is compressed by means of a further compressor V2 and guided as VB2 to reboiler SV1b. In this way, it is possible to completely dispense with the use of external heating steam.

FIG. 4 shows a configuration in which the two streams VB1 and BS1b, after the transfer of energy in the respective reboilers SV1a and SV2a, are run to a flash vessel, where the expansion results in a liquid phase FP1.

FIG. 5 shows a configuration in which there are two additional heat exchangers (WT-1, WT-2) via which energy is transferred from streams VB1 (in WT-1) and VB2 (in WT-2) to streams BS1a (in WT-1) and BS2 (in WT-2).

FIG. 6 shows a configuration in which a condenser (7, 8) for closed-loop control of the pressure is installed in each of the two flash vessels.

### DETAILED DESCRIPTION OF THE INVENTION

This problem is solved by the embodiment of the process proposed in the description herein. Preferred embodiments are also specified in the dependent embodiments. The process according to the invention is a process for separating 1-butene from a raffinate 2 stream containing at least 1-butene, 2-butene, n-butane and isobutane in a separation unit comprising at least two distillation columns DK1 and DK2, wherein
the first distillation column DK1 has at least two reboilers SV1a and SV1b and the second distillation column DK2 has at least one reboiler SV2a;
the reboilers SV1a and SV1b are each fed with a stream which is withdrawn at the lower end of DK1 and, after passing through the respective reboiler, is guided back to DK1;
the reboiler SV2a is fed with a stream which is withdrawn at the lower end of DK2 and, after passing through the respective reboiler, is guided back to DK2; wherein the process comprises the following steps:
(a) the raffinate 2 stream is directed to the first distillation column DK1 and separated in DK1 into at least one vapour stream BS1 which comprises at least 1-butene and isobutane and is withdrawn at the top of DK1, and at least one bottom stream which comprises at least 1-butene and 2-butene and is withdrawn at the bottom of DK1;
(b) the vapour stream is separated into at least two substreams BS1a and BS1b;
(c) a first portion BS1a of the vapour stream BS1 is compressed, giving rise to a compressed stream VB1 relative to the vapour stream BS1a;
(d) energy is transferred from the compressed stream VB1 to the stream in reboiler SV1a;
(e) a portion BS1b of the vapour stream BS1 other than BS1a is conducted to the reboiler SV2a, where energy is transferred from BS1b to the stream in the reboiler SV2a;
(f) streams VB1 and BS1b are directed at least partly to the second distillation column DK2 and separated in DK2 into at least one vapour stream BS2 which comprises at least isobutane and is withdrawn at the top of DK2, and at least one product stream which comprises at least 1-butene and is withdrawn at the bottom of DK2;
(g) the vapour stream BS2 is at least partly compressed, giving rise to a compressed stream VB2 relative to the vapour stream BS2; and
(h) energy is transferred from the compressed stream VB2 to the stream in reboiler SV1b.

One advantage of the process according to the invention is the double thermal integration via the compressed streams VB1 and VB2, by which energy is transferred to the respective streams in the reboilers SV1a and SV1b and hence energy is introduced into the bottoms. The result of the energy transfer in the reboilers SV1a and SV1b in the first distillation column DK1 is that less heating steam, if any at all, has to be used for heating of distillation column DK1. It is thus possible to achieve (almost) complete electrification of the energy-intensive process, which in turn enables the use of green power. This saves considerable amounts of energy costs and CO₂ emission.

According to the present invention, the starting stream from which the 1-butene is to be separated is a raffinate 2 stream containing at least 1-butene, 2-butene, n-butane and isobutane. Corresponding streams are available on the market, for example as a C4 cut from steamcrackers or FCC units. It has already been mentioned in the introduction that raffinate 2 is formed by removal of polyunsaturated C4 hydrocarbons, especially butadiene, and isobutene from the stream. Complete removal is not possible in many cases for economic and technical reasons. However, the amounts of polyunsaturated C4 hydrocarbons, especially butadiene, and isobutene should be at a minimum.

The raffinate 2 used preferably contains less than 1000 ppm, preferably less than 500 ppm, of isobutene. If higher amounts of isobutene are present in the starting stream, it would be possible for an MTBE or ETBE synthesis (methyl tert-butyl ether=MTBE/ethyl tert-butyl ether=ETBE) to be effected between the two distillation columns DK1 and DK2, in order to react the isobutene with methanol (for MTBE) or ethanol (ETBE) and then to separate off the MTBE or ETBE. It is thus possible to greatly reduce the concentration of isobutene upstream of the second distillation column DK2. This is because isobutene would be obtained in the bottoms in the second distillation column and hence in the 1-butene.

Further preferably, the raffinate 2 used in the process of the present invention contains less than 4% by weight of polyunsaturated C4 hydrocarbons. In a particularly preferred embodiment, the concentration of the polyunsaturated C4 hydrocarbons should be less than 500 ppm. If the streams should contain higher amounts of butadiene, it would be possible to conduct a selective hydrogenation beforehand, in which the butadiene is converted to butenes and/or butanes. Corresponding processes are described to the person skilled in the art, for example in EP 3 680 224 A1.

The raffinate 2 stream used may additionally contain certain amounts of water, especially in an amount of 150 to 4000 ppm. It is preferable that the water is at least partly separated off by the process described here. The water will accumulate in the respective vapour stream BS1 and BS2 in each of the two distillation columns DK1 and DK2 and be obtained as a second liquid phase after condensation, which can be separated off via udders in the distillate vessels of DK1 and/or DK2. The bottom products of DK1 and of DK2 feature very low contents of butadiene and water, preferably each below 100 ppm, more preferably below 5 ppm.

The process according to the invention is conducted in a separation unit comprising at least the two distillation columns DK1 and DK2. DK1 is the first distillation column and has at least two reboilers SV1a and SV1b. DK2 is the second distillation column and has at least one reboiler SV2a. In a preferred embodiment, the distillation column has only the two reboilers SV1a and SV1b. It is additionally preferable that the distillation column DK2 has only the one reboiler SV2a. The pressures in the two distillation columns DK1 and DK2 should in particular have to be chosen such that heat can be transferred in the reboilers. The terms “first distillation column”, “distillation column DK1” and “DK1” should be considered to be synonymous in the context of the present invention. The terms “second distillation column”, “distillation column DK2” and “DK2” should likewise be considered to be synonymous in the context of the present invention.

The energy needed for the separation task is introduced into the first distillation column DK1 via the reboilers SV1a and SV1b. The reboilers SV1a and SV1b are each fed with a stream which is withdrawn at the lower end of DK1 and, after passing through the respective reboiler, is guided back to DK1. The respective stream is heated as it passes through the reboiler SV1a or SV1b. One option is for the two streams to be withdrawn independently, i.e. at two different sites in the bottom of DK1. It is alternatively possible to withdraw only one stream which is then separated into the two streams, optionally with a split controller by means of which the mass flows of the two streams are adjusted with reference to a predetermined parameter. The feeds of the two streams from the two reboilers SV1a and SV1b are especially at different points in the bottom, meaning that the two streams, after passing through the reboiler, are not mixed prior to entry into DK2.

The situation is comparable for the reboiler SV2a of the second distillation column DK2, by means of which the energy needed for the separation task is introduced. For this purpose, the reboiler SV2a is fed with a stream which is withdrawn at the lower end of DK2 and, after passing through the respective reboiler, is guided back to DK2. The stream is heated as it passes through the reboiler SV2a and at least partly evaporates as it does so.

According to the invention, “reboilers” refer to evaporators that heat the bottom of the respective distillation column. Such a reboiler is typically disposed outside the respective distillation column. Since reboilers transfer energy, in particular heat, from one stream to another, they are heat transferrers. The stream to be evaporated is drawn off from the bottom of the distillation column via a draw and fed to the reboiler. The evaporated stream, with or without a residual proportion of liquid, is returned back to the respective distillation column in the region of the bottom via at least one feed.

Suitable evaporators that can be used as reboilers are, for example, natural circulation evaporators, forced circulation evaporators, forced circulation flash evaporators, kettle evaporators, falling-film evaporators or thin-film evaporators. Heat exchangers for the evaporator that are typically used in the case of natural circulation evaporators and forced circulation evaporators are a shell-and-tube or plate apparatus. In addition to those mentioned, it is alternatively possible to use any other design of evaporator which is known to those skilled in the art and is suitable for use in a distillation column.

The raffinate 2 which is directed to the first distillation column DK1 is separated in distillation column DK1 into at least two streams, i.e. at least one vapour stream BS1 which comprises at least 1-butene and isobutane and is withdrawn at the top of DK1, and at least one bottom stream which comprises at least 1-butene and 2-butene and is withdrawn at the bottom of DK1. This bottom stream can be guided to an oligomerization (not shown). The vapour stream BS1 can also be withdrawn at the top of the distillation column in the form of multiple substreams BS1n, where n is an integer and is equal to the number of substreams. The same also applies to the bottom stream. The temperature at the bottom of the first distillation column DK1 is preferably in the range from 40 to 110° C., preferably 50 to 100° C.

In principle, the raffinate 2 stream can be directed into the first distillation column DK1 via one or more feed points. If there are multiple feed points for the raffinate 2 stream, multiple separate streams are accordingly directed into the distillation column. In the embodiments of the present invention in which the raffinate 2 stream is directed into the distillation column DK1 as two or more separate streams, it is advantageous when the feed points of the individual streams are essentially at the same height on the distillation column DK1.

The pressure and temperature of the vapour stream BS1 are specified hereinafter. This relates in particular to the pressure and temperature of the at least one vapour stream BS1 when it is withdrawn from distillation column DK1. The pressure of the vapour stream BS1 is especially in the range from 6 to 15 bar absolute, preferably in the range from 7.5 to 13 bar absolute. The temperature of the vapour stream BS1 is especially in the range from 45° C. to 120° C., preferably in the range from 48° C. to 100° C., further preferably in the range from 50° C. to 90° C., further preferably in the range from 55° C. to 80° C., more preferably in the range from 60°° C. to 80° C.

The distillation column DK1 used for the separation of the raffinate 2 stream may be any distillation column known to the person skilled in the art. Distillation column DK1 preferably contains internals. Suitable internals are for example trays, unstructured packings (random packings) or structured packings. Trays used are typically bubble-cap trays, sieve trays, valve trays, tunnel-cap trays or slotted trays. Unstructured packings are generally beds of random packings. Random packing elements used are typically Raschig rings, Pall rings, Berl saddles or Intalox® saddles. Structured packings are sold, for example, under the Sulzer Mellapack® trade name. In addition to the internals mentioned, further suitable internals are known to those skilled in the art and may likewise be used.

Preferred internals have a low specific pressure drop per theoretical plate. Structured packings and random packing elements have, for example, a significantly lower pressure drop per theoretical plate than trays. This has the advantage that the pressure drop in distillation column DK1 remains as low as possible and hence the mechanical output of the compressor and the temperature of the raffinate 2 stream to be evaporated remains low.

In a particularly preferred embodiment of the present invention, distillation column DK1 comprises a multitude of trays, preferably between 150 and 300 trays, further preferably between 170 and 220 trays.

What is meant in the context of the present invention by the withdrawal of at least one vapour stream BS1 comprising at least 1-butene and isobutane at the top of distillation column DK1 is in particular that the at least one vapour stream BS1 is withdrawn as top stream or as side draw above the internals in distillation column DK1.

What is meant in the context of the present invention by the withdrawal of the at least one vapour stream comprising at least 1-butene and 2-butene at the bottom of distillation column DK1 is in particular that the at least one bottom stream is withdrawn directly at the bottom or at the lower tray of distillation column DK1.

Distillation column DK1 is still operated with reflux, preferably with reflux. What is meant by “reflux” is that the vapour stream BS1 withdrawn at the top end of distillation column DK1 is at least partly fed back to distillation column DK1. In the cases where such a reflux is established, the reflux ratio is preferably 2 to 30, more preferably 5 to 20, especially preferably 8 to 15.

A reflux can be established by mounting a condenser at the top of distillation column DK1. Vapour stream BS1 is partly condensed in the condenser and fed back to distillation column DK1. The vapour stream or a portion thereof can also be applied as reflux to the distillation column only after compression and expansion. In general and in the context of the present invention, a reflux ratio means the ratio of the proportion of the mass flow rate (kg/h) withdrawn from the column that is returned back into the column in liquid form (reflux) to the proportion of that mass flow rate (kg/h) which is discharged from the respective column in liquid form or gaseous form.

After the withdrawal of vapour stream BS1, the vapour stream BS1 is separated in step (b) into at least two substreams BS1a and BS1b. The separation can in principle be effected in a known manner, for example by means of a splitter (with closed-loop control using a compressor and/or an adjuster valve). Another conceivable option here would be closed-loop control via which the mass flow rates of BS1a and BS1b are adjusted as a function of a particular parameter.

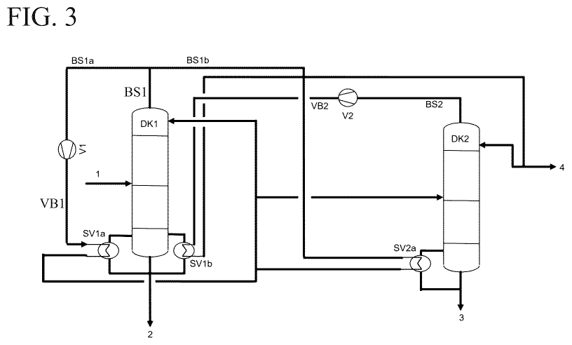

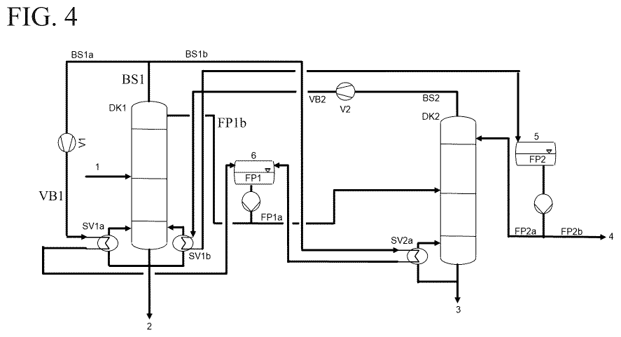

Subsequently, in step (c), a first portion BS1a of the vapour stream BS1 is compressed, giving rise to a compressed stream VB1 relative to vapour substream BS1a. Stream BS1a generally has the same pressure as stream BS1 when it is withdrawn from distillation column DK1. It may be advantageous to heat stream BS1a prior to compression in order that no biphasic mixture is formed on compression. The heating can be conducted by means of internal (see WT-1 in FIGS. 3 and 4 of the present application) or external heat sources. The pressure of VB1 after the compression is higher than the pressure of BS1a. The exact value for the pressure of VB1 can be set by the person skilled in the art depending on the requirements in the subsequent energy transfer, provided that the condition that pressure VB1 >pressure BS1a is met. The quotient of pressure VB1/pressure BS1a (pressures each in bar abs.) is preferably in the range from 1.1 to 10, more preferably 1.2 to 8, more preferably 1.25 to 7, most preferably 1.3 to 6.

The temperature of substream VB1 is preferably higher than the temperature of vapour substream BS1a, and the quotient of temperature VB1/temperature BS1a (temperature in each case in K) is preferably in the range from 1.03 to 10, more preferably 1.04 to 9, more preferably 1.05 to 8, more preferably 1.06 to 7, more preferably 1.07 to 6, most preferably 1.08 to 5.

The at least one portion of vapour substream BS1a can be compressed in step (c) in any desired manner known to the person skilled in the art. For example, the compression can be performed mechanically and in a single-stage or multistage compression. What is meant by “single-stage” in this connection is that compression takes place from one pressure level to another. What is meant by “multistage” is that compression is effected first to a pressure level X and then from X to the pressure level Y. In a multistage compression, it is possible to use two or more compressors of the same type or compressors of different types. A multistage compression can be effected with one or more compressors. The use of single-stage compression or multistage compression depends on the compression ratio and hence on the pressure to which the vapour substream BS1a is to be compressed.

A suitable compressor in the process according to the invention, especially for compression of the vapour streams BS1a to VB1, is any compressor known to the person skilled in the art, preferably a mechanical compressor, with which gas streams can be compressed. Suitable compressors are, for example, single- stage or multistage geared turbocompressors, piston compressors, screw compressors, centrifugal compressors or axial compressors.

In step (d) of the process according to the invention, energy is transferred from the compressed stream VB1 to the stream in reboiler SV1a. Step (d) lowers the energy of VB1, such that VB1 is especially at least partly condensed. According to the invention, the phrase transfer of energy especially means heating, i.e. transfer of energy in the form of heat.

The transfer of energy from VB1 to the stream in the reboiler SV1a, preferably the heating of the stream in the reboiler SV1a by VB1, is preferably direct. What is meant by direct transfer is that, although VB1 and the stream in SV1a do not come into direct contact, energy, especially heat, is transferred from VB1 to the stream in SV1a without the presence of any additional heat transfer medium. Reboilers SV1a used may be the heat transferrers or heat exchangers that are familiar to the person skilled in the art, especially evaporators.

In a preferred embodiment, step (d) of the process according to the invention may lead to a particular benefit. Rather than the surplus energy obtained on compression of the vapour stream BS1a to the compressed vapour stream VB1 being dissipated unutilized here, it is used in the distillation DK2. This is effected in that BS1a is first compressed to VB1 in such a way that VB1 is compressed beyond the degree required in SV1a. The heat of condensation which is obtained in the additional compression can be fed into column DK2 via stream VB1. The required additional compressor output is generally less than the heating steam power saved thereby.

In step (e) of the process according to the invention, at least a portion BS1b of the vapour stream BS1 other than BS1a is conducted to the reboiler SV2a, where energy is transferred in the reboiler SV2a from BS1b to the stream present in the reboiler. Step (e) lowers the energy of BS1b, such that BS1b is especially at least partly condensed.

The transfer of energy from BS1b to the stream in the reboiler SV2a, preferably the heating of the stream in the reboiler SV2a by BS1b, is preferably direct. What is meant by direct transfer is that, although BS1b and the stream in SV2a do not come into direct contact, energy, especially heat, is transferred from BS1b to the stream in SV2a without the presence of any additional heat transfer medium. Reboilers SV2a used may be the heat transferrers or heat exchangers that are familiar to the person skilled in the art, especially evaporators.

Once streams VB1 and BS1b have passed through the reboiler SV1a or SV2a and transferred energy to the respective streams, these streams VB1 and BS1b, in step (f), are at least partly directed to the second distillation column DK2, where the separation between isobutane and 1-butene is then effected in order to obtain a 1-butene stream of maximum purity. The two streams VB1 and BS1b may independently be guided as separate feed streams or together to the second distillation column DK2.

Before the streams are guided to the second distillation column in step (f) to the second distillation column, streams VB1 and BS1b, in a preferred embodiment, are guided to a flash vessel and expanded therein to obtain a liquid phase FP1 of streams VB1 and BS1b. The flash vessel may additionally comprise a condenser in order to condense a portion of the gaseous phase obtained.

In a preferred embodiment of the present invention, streams VB1 and BS1b are guided collectively to the second distillation column. For this purpose, the two streams are in particular brought to the same pressure and the same temperature. Thus, if streams VB1 and BS1b are to be conducted collectively to the distillation column, it is preferable that streams VB1 and BS1b are combined in the flash vessel and are obtained as a common liquid phase FP1.

At least a portion FP1a of the liquid phase FP1 is then directed in step (f) to distillation column DK2. For this purpose, in a particularly preferred embodiment, a pump is used. It is possible here to use pumps known to the person skilled in the art. Suitable pumps are, for example, standard chemical pumps.

It is further preferable that a portion FP1b of the liquid phase FP1 other than FP1a is returned as reflux to the first distillation column DK1. It is particularly preferable that this transfers energy from stream FP1b to the raffinate 2 stream before the raffinate 2 stream is introduced into the first distillation column DK1. This preheats the raffinate 2 stream. This is energetically advantageous because less energy has to be introduced for the separation task via the reboilers. The transfer of energy from FP1b to the raffinate 2 stream, preferably the heating of the raffinate 2 stream by FP1b, is preferably direct, i.e. without use of a (further) heat transfer medium. For this purpose, it is possible to use heat transferrers or heat exchangers that are familiar to the person skilled in the art.

In the second distillation column DK2, the streams that both respectively comprise at least isobutane and 1-butene are separated into at least one vapour stream BS2 which comprises at least isobutane and is withdrawn at the top of DK2, and at least one product stream which comprises at least 1-butene and is withdrawn at the bottom of DK2.

The distillation column DK2 used for the separation of the two streams VB1 and BS1b may be any distillation column known to the person skilled in the art. Distillation column DK2 preferably contains internals. Suitable internals are for example trays, unstructured packings (random packings) or structured packings. Trays used are typically bubble-cap trays, sieve trays, valve trays, tunnel-cap trays or slotted trays. Unstructured packings are generally beds of random packings. Random packing elements used are typically Raschig rings, Pall rings, Berl saddles or Intalox® saddles. Structured packings are sold, for example, under the Sulzer Mellapack® trade name. In addition to the internals mentioned, further suitable internals are known to those skilled in the art and may likewise be used.

Preferred internals have a low specific pressure drop per theoretical plate. Structured packings and random packing elements have, for example, a significantly lower pressure drop per theoretical plate than trays. This has the advantage that the pressure drop in distillation column DK2 remains as low as possible and hence the mechanical output of the compressor and the temperature of the two streams VB1 and BS1b to be evaporated remains low.

In a particularly preferred embodiment of the present invention, the second distillation column DK2 comprises a multitude of trays, preferably between 150 and 300 trays, further preferably between 170 and 220 trays.

What is meant in the context of the present invention by the withdrawal of at least one vapour stream BS2 comprising at least isobutane at the top of distillation column DK2 is in particular that the at least one vapour stream BS2 is withdrawn as top stream or as side draw above the internals in distillation column DK2.

What is meant in the context of the present invention by the withdrawal of the at least one product stream comprising at least 1-butene at the bottom of distillation column DK2 is in particular that the at least one product stream is withdrawn directly at the bottom or at the lower tray of distillation column DK2. The product stream preferably contains at least 99% by weight of 1-butene, further preferably at least 99.5% by weight of 1-butene, more preferably at least 99.6% by weight of 1-butene. The 1-butene is the target product of the present process, and therefore the product stream is discharged from the process. The 1-butene may be used for example as a comonomer in the production of polyethylene.

It should be noted here that the separation sharpness in the first distillation column DK1 ultimately determines the purity of the 1-butene in the product stream which is withdrawn from the second distillation column DK2. This is because butane is likewise obtained as a high boiler in DK2, and therefore with the 1-butene. This would contaminate the 1-butene. It should thus be ensured that the separation of the raffinate 2 stream in DK1 is run as far as possible such that barely any butane gets to DK2. It is therefore preferable that the stream guided to DK2 (1a, FP1a) contains not more than between 500 and 900 ppm of butanes, based on the total amount of the stream.

The temperature at the bottom of the second distillation column DK2 during the process according to the invention is preferably in the range from 30 to 100° C., preferably 45 to 80° C. Further preferably, the pressure at the top of the second distillation column DK2 is in the range from 3 to 12 bar absolute, preferably 5 to 10 bar absolute.

Distillation column DK2 can also be operated with reflux. What is meant by reflux is that the vapour stream BS2 withdrawn at the top end of distillation column DK2 is at least partly fed back to distillation column DK2. In the cases where such a reflux is established, the reflux ratio is preferably 10 to 80, especially preferably 30 to 50.

A reflux can be established by mounting a condenser at the top of distillation column DK2. Vapour stream BS2 is partly condensed in the condenser and fed back to distillation column DK2. In general and in the context of the present invention, a reflux ratio means the ratio of the proportion of the mass flow rate (kg/h) withdrawn from the column that is returned back into the column in liquid form (reflux) to the proportion of that mass flow rate (kg/h) which is discharged from the respective column in liquid form or gaseous form.

Subsequently, in step (g), the vapour stream BS2 is at least partly compressed, giving rise to a compressed stream VB2 relative to the vapour stream BS2. The pressure of VB2 after the compression is higher than the pressure of BS2. The exact value for the pressure of VB2 can be set by the person skilled in the art depending on the requirements in the subsequent energy transfer, provided that the condition that pressure VB2 >pressure BS2 is met. The quotient of pressure VB2/pressure BS2 (pressures each in bar abs.) is preferably in the range from 1.1 to 10, more preferably 1.2 to 8, more preferably 1.25 to 7, most preferably 1.3 to 6. It may be advantageous to heat stream BS2 prior to compression in order that no biphasic mixture is formed on compression. The heating can be conducted by means of internal (see WT-2 in FIGS. 3 and 4 of the present application) or external heat sources.

The temperature of substream VB2 is preferably higher than the temperature of vapour stream BS2, and the quotient of temperature VB2/temperature BS2 (temperature in each case in K) is preferably in the range from 1.03 to 10, more preferably 1.04 to 9, more preferably 1.05 to 8, more preferably 1.06 to 7, more preferably 1.07 to 6, most preferably 1.08 to 5.

The at least one portion of vapour substream BS2 can be compressed in step (c) in any desired manner known to the person skilled in the art. For example, the compression can be performed mechanically and in a single-stage or multistage compression. In a multistage compression, it is possible to use two or more compressors of the same type or compressors of different types. A multistage compression can be effected with one or more compressors. The use of single-stage compression or multi-stage compression depends on the compression ratio and thus on the pressure to which the vapour substream BS2 is to be compressed.

A suitable compressor in the process according to the invention, especially for compression of the vapour substream B2 to VB2, is any compressor known to the person skilled in the art, preferably a mechanical compressor, with which gas streams can be compressed. Suitable compressors are, for example, single- stage or multistage turbines, piston compressors, screw compressors, centrifugal compressors or axial compressors.

In step (h) of the process according to the invention, energy is transferred from the compressed stream VB2 to the stream in reboiler SV1b. Step (d) lowers the energy of VB2, such that VB2 is especially at least partly condensed. According to the invention, the phrase transfer of energy especially means heating, i.e. transfer of energy in the form of heat.

The transfer of energy from VB2 to the stream in the reboiler SV1b, preferably the heating of the stream in the reboiler SV1b by VB2, is preferably direct. What is meant by direct transfer is that, although VB2 and the stream in SV1b do not come into direct contact, energy, especially heat, is transferred from VB2 to the stream in SV1b without the presence of any additional heat transfer medium. Reboilers SV1b used may be the heat transferrers or heat exchangers that are familiar to the person skilled in the art, especially evaporators.

Once stream VB2 has passed through reboiler SV1b and transferred energy to the stream present therein, VB2 can be discharged from the process as isobutane stream IB1. However, before VB2 is removed from the process as IB1, stream VB2, in a preferred embodiment of the present invention, is guided to a flash vessel and expanded therein to obtain a liquid phase FP2. The flash vessel may additionally comprise a condenser in order to condense a portion of the gaseous phase obtained.

At least a portion FP2a of the liquid phase FP2 can then be discharged from the process as isobutane stream IB1. For this purpose, in a particularly preferred embodiment, a pump is used. Under some circumstances and given a sufficient pressure ratio, it would also be possible that no pump is used. If a pump is used, it is possible to use pumps known to the person skilled in the art. Suitable pumps are, for example, standard chemical pumps. It is further preferable that a portion FP2b of the liquid phase FP1 other than FP2a is returned as reflux to the first distillation column DK2.

In the basic configuration of the present invention, the two streams BS1a and BS2 are each compressed with a single compressor. In a preferred embodiment of the present invention, the two streams BS1a and BS2 are compressed in a single, preferably multistage, compressor. The number of stages necessary depends on the target compression ratio.

The thermal integration by means of the vapour compression described here would also be combinable with other measures for thermal integration. It would also be possible here to provide one or more heat pump(s).

The present invention is elucidated hereinbelow with reference to figures. The figures are for illustration but are not to be understood as limiting.

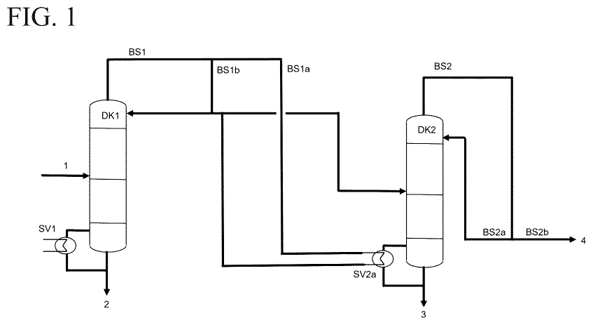

FIG. 1 shows a configuration according to the prior art. The raffinate 2 stream (1) is directed to the first distillation column DK1, where it is separated into a vapour stream BS1 which comprises at least 1-butene and isobutane and is withdrawn at the top of DK1, and a bottom stream (2) which comprises at least 1-butene and 2-butene and is withdrawn at the bottom of DK1. This stream (2) can be conducted to an oligomerization (not shown). The vapour stream BS1 is separated into the two vapour streams BS1a and BS1b, optionally after prior condensation (not shown). BS1a is conducted as reflux to the first distillation column DK1. BS1b is directed to reboiler SV2a of the second distillation column without additional compression, where it transfers energy to the stream present therein, which is heated coming from the bottom and then returned again. This corresponds to a conventional dual-pressure circuit in which DK1 is operated at a higher pressure than DK2 (see also Example 1). In distillation column 2, the streams are separated into a vapour stream BS2 which comprises at least isobutane and is withdrawn at the top of DK2, and a product stream (3) which comprises at least 1-butene and is withdrawn at the bottom of DK2. The vapour stream is separated into streams B2a and B2b after optional condensation. B2a goes back to DK2 as reflux and B2b is discharged from the process as isobutane stream (4).

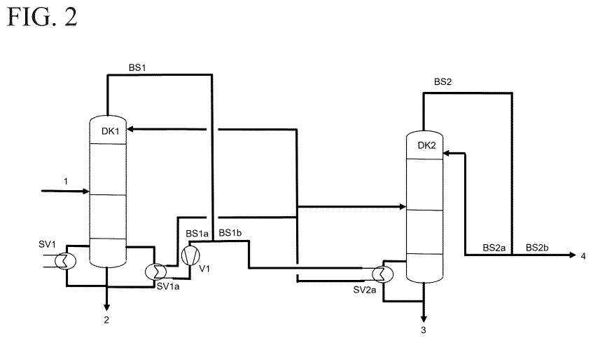

FIG. 2 shows a likewise non-inventive embodiment which corresponds very substantially to the configuration according to FIG. 1. The sole difference is that stream BS1a is brought to a higher pressure with compressor V1 and then directed to reboiler SV1a for energy transfer. The two streams BS1a and BS1b are combined after the energy transfer and go partly to DK1 as reflux and partly to DK2 as feed.

FIG. 3 shows an embodiment in accordance with the invention which is identical in large parts to the embodiment shown in FIG. 2. The difference is that the stream compressed with compressor V1 is called VB1. In addition, vapour stream BS2 is compressed by means of a further compressor V2 and guided as VB2 to reboiler SV1b. In this way, it is possible to completely dispense with the use of external heating steam.

FIG. 4 shows an embodiment which is likewise in accordance with the invention and corresponds largely to FIG. 3. The difference is that the two streams VB1 and BS1b, after the transfer of energy in the respective reboilers SV1a and SV2a, are run to a flash vessel, where the expansion results in a liquid phase FP1. This liquid phase is separated into the two streams FP1a, which is conducted to the second distillation column DK2, and FP1b, which is directed as reflux to the first distillation column. The sequence is comparable for stream VB2 after the transfer of energy in the reboiler SV1b. VB1 then arrives in a flash vessel and condenses at least partly as liquid phase FP2, from which a portion FP2a from isobutane stream (4) leaves the process and another portion FP2b is returned to the second distillation column as reflux.

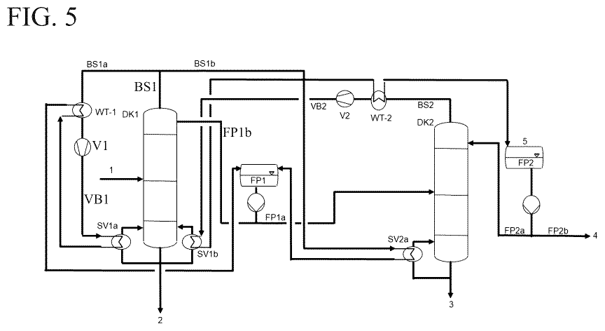

FIG. 5 shows an embodiment which is largely identical to the embodiments represented in FIG. 1 and FIG. 2. The difference is that there are two additional heat exchangers (WT-1, WT-2) via which energy is transferred from streams VB1 (in WT-1) and VB2 (in WT-2) to streams BS1a (in WT-1) and BS2 (in WT-2). This can additionally save energy.

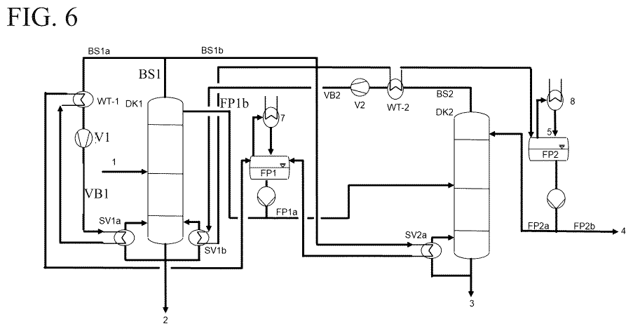

FIG. 6 shows an embodiment which is largely identical to the embodiments represented in FIG. 1 to FIG. 3. The difference is that a condenser (7, 8) for closed-loop control of the pressure is installed in each of the two flash vessels. In addition, the condenser can also be used for the startup process.

### EXAMPLES

For all the examples that follow, a raffinate 2 stream of 55 t/h was used. The raffinate 2 stream has the following composition: 1-butene 45.1%/n-butane 22.4%/trans-2-butene 15.9%/cis-2-butene 8.9%/isobutane 7.4%/isobutene 45 ppm and water 590 ppm.

The amount of energy needed for operation of the plants detailed in the examples for separation of 1-butene from raffinate 2 was calculated by a simulation using Aspen V10. The physical data were validated by operational data and operational trials.

### Example 1 (Non-Inventive)

In the embodiment according to FIG. 1, exclusively direct thermal integration between column DK1 and DK2 was implemented (as disclosed in DE 10 2005 062 700 A1). In this case, column DK1 is operated at a top pressure of 11 bar and column DK2 at a top pressure of 7 bar. It is thus possible to use a portion of vapour stream BS1b to transfer heat to DK2 via reboiler SV2a. The amount of heat required for a 1-butene volume of 10.3 t/h at a purity of 99.6% 1-butene is 10.3 MW in DK2. For this purpose, a vapour stream of 125.5 t/h from DK1 is used to heat DK2. Nevertheless, it is necessary to introduce 15.9 MW in DK1 via an external heat source/medium (e.g. heating steam).

### Example 2 (Non-Inventive)

In this embodiment, direct thermal integration between column DK1 and DK2 was implemented (as disclosed in DE 10 2005 062 700 A1) and was supplemented with a vapour compression (see FIG. 2). The heat of condensation of 4.0 MW from DK1 which is not used in Example 1 for the thermal integration between DK1 and DK2 is rendered utilizable via a one-stage vapour compression via compressor V1 and introduced via a secondary reboiler SV1a in DK1. It is possible to transfer a total of 4.4 MW through the vapour compression via reboiler SV1a. Accordingly, the external heat demand in column DK1 is reduced from 15.9 MW (Example 1) to 11.5 MW. A total of 382 KW of electrical power has to be expended in compressor V1 in order to compress the vapour from 11 bar to 16.9 bar, in order thus to create a driving temperature differential in SV1a of 8 K.

### Example 3 (Inventive)

In this embodiment, direct thermal integration between column DK1 and DK2 was implemented (as disclosed in DE 10 2005 062 700 A1) and was supplemented in accordance with the invention with a multistage vapour compression (see FIG. 3). The embodiment which is described in Example 2 is supplemented by a further vapour compression. The vapour in DK2 is compressed in order to render the heat of condensation of 9.9 MW from DK2 utilizable for DK1. The heat of condensation that has been upgraded by means of compressor V2 is transferred via a further reboiler SV1b. For this purpose, the vapour stream in the second column DK2 is compressed from 7 bar to 21 bar. An electrical power of 2.6 MW is required. It is possible to transfer a total of 11.5 MW via SV1b, such that no external heat source is required for steady-state operation. Accordingly, the energy-intensive process of 1-butene distillation has been fully electrified.

The results of Examples 1 to 3 are summarized below in Table 1.

TABLE 1
Summary of examples

| | Example | Example | Example |
|---|---|---|---|
| | 1 | 2 | 3* |
| External heating power [MW] | 15.9 | 11.5 | 0 |
| Electrical power for | 0 | 0.38 | 2.98 |
| compressor [MW] | | | |

*Inventive

It is found that the inventive configuration of the process has the effect that much less external heating power has to be used than in the known solution. The savings potential is thus considerable. The electrical power additionally required for operation of the compressors is much smaller and can enable CO₂-neutral operation when green power is used.

1. A process for separating 1-butene from a hydrocarbon stream containing at least 1-butene, 2-butene, n-butane and isobutane in a separation unit comprising a first distillation column DK1 and a second distillation column DK2, wherein
the first distillation column DK1 has at least two reboilers SV1a and SV1b and the second distillation column DK2 has at least one reboiler SV2a;
the at least two reboilers SV1a and SV1b are each fed with a stream which is withdrawn at a lower end of the first distillation column DK1 and, after passing through a respective reboiler, is guided back to the first distillation column DK1;
the at least one reboiler SV2a is fed with a stream which is withdrawn at a lower end of the second distillation column DK2 and, after passing through a respective reboiler, is guided back to the second distillation column DK2; the process comprising:
(a) directing the raffinate 2 stream to the first distillation column DK1 and separating the raffinate 2 stream in the first distillation column DK1 into at least one vapour stream BS1 which comprises at least 1-butene and isobutane and is withdrawn at a top of the first distillation column DK1, and at least one bottom stream which comprises at least 1-butene and 2-butene and is withdrawn at a bottom of the first distillation column DK1;
(b) separating the at least one vapour stream BS1 into at least two substreams comprising a substream BS1a and a substream BS1b;
(c) compressing a first portion of substream BS1a of the at least one vapour stream BS1, giving rise to a compressed stream VB1 relative to the vapour stream BS1a;
(d) transferring energy from the compressed stream VB1 to a stream in reboiler SV1a;
(e) conducting a portion of substream BS1b of the at least one vapour stream BS1 other than substream BS1a to the at least one reboiler SV2a, where energy is transferred from substream BS1b to a stream in the at least one reboiler SV2a;
(f) directing compressed stream VB1 and substream BS1b at least partly to the second distillation column DK2 and separating the compressed stream VB1 and the substream BS1b in the second distillation column DK2 into at least one vapour stream BS2 which comprises at least isobutane and is withdrawn at a top of the second distillation column DK2, and at least one product stream which comprises at least 1-butene and is withdrawn at a bottom of the second distillation column DK2;
(g) at least partly compressing the at least one vapour stream BS2, giving rise to a compressed stream VB2 relative to the at least one vapour stream BS2; and
(h) transferring energy from the compressed stream VB2 to the stream in reboiler SV1b.

2. The process according to claim 1, further comprising:
guiding the compressed stream VB1 and the substream BS1b to a flash vessel, where they are expanded, resulting in a liquid phase FP1 of the substream BS1a und the substream BS1b.

3. The process according to claim 2, further comprising:
combining and obtaining the compressed stream VB1 and the substream BS1b in the flash vessel as a common liquid phase FP1.

4. The process according to claim 3, further comprising:
directing at least a portion FP1a of the common liquid phase FP1 to the second distillation column DK2.

5. The process according to claim 3, further comprising:
returning another portion FP1b of the common liquid phase FP1 as reflux to the first distillation column DK1.

6. The process according to claim 5, further comprising:
transferring energy from stream FP1b to the raffinate 2 stream before the raffinate 2 stream is introduced into the first distillation column DK1.

7. The process according to claim 1, further comprising:
adjusting a temperature in the bottom of the first distillation column DK1 to a range from 40 to 110° C.

8. The process according to claim 1, further comprising:
adjusting a temperature in the bottom of the second distillation column DK2 to a range from 30 to 100° C.

9. The process according to claim 1, wherein the first distillation column DK1 with between 150 and 300 plates.

10. The process according to claim 1, wherein the second distillation column DK2 with between 150 and 300 plates.

11. The process according to claim 1, further comprising:
employing only a single compressor for the compression of streams BS1a and BS2.

12. The process according to claim 1, further comprising:
expanding the compressed stream VB2 into a flash vessel, where a liquid phase FP2 is obtained.

13. The process according to claim 12, further comprising:
discharging at least a portion FP2a of the liquid phase FP2 from the process as product stream.

14. The process according to claim 13, further comprising:
returning another portion FP2b of the liquid phase FP2 as reflux to the second distillation column DK2.

15. The process according to claim 1, wherein the product stream contains at least 99% by weight of 1-butene.
